SEQUENCE LISTING (30 sequences)

SEQ ID NO: 1 - DNA, 1560 bp, Homo sapiens
  source 1..1560: mol_type = genomic DNA
atggatgctgatgagggtcaagacatgtcccaagtttcagggaaggaaagcccccctgtaagcgatactccagatgagggcgatgagcccatgccgatccccgaggacctctccaccacctcgggaggacagcaaagctccaagagtgacagagtcgtggccagtaatgttaaagtagagactcagagtgatgaagagaatgggcgtgcctgtgaaatgaatggggaagaatgtgcggaggatttacgaatgcttgatgcctcgggagagaaaatgaatggctcccacagggaccaaggcagctcggctttgtcgggagttggaggcattcgacttcctaacggaaaactaaagtgtgatatctgtgggatcatttgcatcgggcccaatgtgctcatggttcacaaaagaagccacactggagaacggcccttccagtgcaatcagtgcggggcctcattcacccagaagggcaacctgctccggcacatcaagctgcattccggggagaagcccttcaaatgccacctctgcaactacgcctgccgccggagggacgccctcactggccacctgaggacgcactccgttggtaaacctcacaaatgtggatattgtggccgaagctataaacagcgaagctctttagaggaacataaagagcgctgccacaactacttggaaagcatgggccttccgggcacactgtacccagtcattaaagaagaaactaatcacagtgaaatggcagaagacctgtgcaagataggatcagagagatctctcgtgctggacagactagcaagtaacgtcgccaaacgtaagagctctatgcctcagaaatttcttggggacaagggcctgtccgacacgccctacgacagcagcgccagctacgagaaggagaacgaaatgatgaagtcccacgtgatggaccaagccatcaacaacgccatcaactacctgggggccgagtccctgcgcccgctggtgcagacgcccccgggcggttccgaggtggtcccggtcatcagcccgatgtaccagctgcacaagccgctcgcggagggcaccccgcgctccaaccactcggcccaggacagcgccgtggagaacctgctgctgctctccaaggccaagttggtgccctcggagcgcgaggcgtccccgagcaacagctgccaagactccacggacaccgagagcaacaacgaggagcagcgcagcggtctcatctacctgaccaaccacatcgccccgcacgcgcgcaacgggctgtcgctcaaggaggagcaccgcgcctacgacctgctgcgcgccgcctccgagaactcgcaggacgcgctccgcgtggtcagcaccagcggggagcagatgaaggtgtacaagtgcgaacactgccgggtgctcttcctggatcacgtcatgtacaccatccacatgggctgccacggcttccgtgatccttttgagtgcaacatgtgcggctaccacagccaggaccggtacgagttctcgtcgcacataacgcgaggggagcaccgcttccacatgagctaa

SEQ ID NO: 2 - DNA, 1530 bp, Homo sapiens
  source 1..1530: mol_type = genomic DNA
atggaagatatacaaacaaatgcggaactgaaaagcactcaggagcagtctgtgcccgcagaaagtgcagcggttttgaatgactacagtttaaccaaatctcatgaaatggaaaatgtggacagtggagaaggcccagccaatgaagatgaagacataggagatgattcaatgaaagtgaaagatgaatacagtgaaagagatgagaatgttttaaagtcagaacccatgggaaatgcagaagagcctgaaatcccttacagctattcaagagaatataatgaatatgaaaacattaagttggagagacatgttgtctcattcgatagtagcaggccaaccagtggaaagatgaactgcgatgtgtgtggattatcctgcatcagcttcaatgtcttaatggttcataagcgaagccatactggtgaacgcccattccagtgtaatcagtgtggggcatcttttactcagaaaggtaacctcctccgccacattaaactgcacacaggggaaaaaccttttaagtgtcacctctgcaactatgcatgccaaagaagagatgcgctcacggggcatcttaggacacattctgtggagaaaccctacaaatgtgagttttgtggaaggagttacaagcagagaagttcccttgaggagcacaaggagcgctgccgtacatttcttcagagcactgacccaggggacactgcaagtgcggaggcaagacacatcaaagcagagatgggaagtgaaagagctctcgtactggacagattagcaagcaatgtggcaaaacgaaaaagctcaatgcctcagaaattcattggtgagaagcgccactgctttgatgtcaactataattcaagttacatgtatgagaaagagagtgagctcatacagacccgcatgatggaccaagccatcaataacgccatcagctatcttggcgccgaagccctgcgccccttggtccagacaccgcctgctcccacctcggagatggttccagttatcagcagcatgtatcccatagccctcacccgggctgagatgtcaaacggtgcccctcaagagctggaaaagaaaagcatccaccttccagagaagagcgtgccttctgagagaggcctctctcccaacaatagtggccacgactccacggacactgacagcaaccatgaagaacgccagaatcacatctatcagcaaaatcacatggtcctgtctcgggcccgcaatgggatgccacttctgaaggaggttccccgctcttacgaactcctcaagcccccgcccatctgcccaagagactccgtcaaagtgatcaacaaggaaggggaggtgatggatgtgtatcggtgtgaccactgccgcgtcctcttcctggactatgtgatgttcacgattcacatgggctgccacggcttccgtgaccctttcgagtgtaacatgtgtggatatcgaagccatgatcggtatgagttctcgtctcacatagccagaggagaacacagagccctgctgaagtga

SEQ ID NO: 3 - DNA, 22 bp, synthetic construct
  misc_feature 1..22: note = Human GAPDH F primer
  source 1..22: mol_type = other DNA
gtctcctctgacttcaacagcg

SEQ ID NO: 4 - DNA, 22 bp, synthetic construct
  misc_feature 1..22: note = Human GAPDH R primer
  source 1..22: mol_type = other DNA
accaccctgttgctgtagccaa

SEQ ID NO: 5 - DNA, 23 bp, synthetic construct
  misc_feature 1..23: note = Human IKZF1 F primer
  source 1..23: mol_type = other DNA
gctgccacaactacttggaaagc

SEQ ID NO: 6 - DNA, 22 bp, synthetic construct
  misc_feature 1..22: note = Human IKZF1 R primer
  source 1..22: mol_type = other DNA
agtctgtccagcacgagagatc

SEQ ID NO: 7 - DNA, 22 bp, synthetic construct
  misc_feature 1..22: note = Human IKZF3 F primer
  source 1..22: mol_type = other DNA
gccaaagaagagatgcgctcac

SEQ ID NO: 8 - DNA, 22 bp, synthetic construct
  misc_feature 1..22: note = Human IKZF3 R primer
  source 1..22: mol_type = other DNA
ggcgttattgatggcttggtcc

SEQ ID NO: 9 - DNA, 23 bp, synthetic construct
  misc_feature 1..23: note = Mouse GAPDH F primer
  source 1..23: mol_type = other DNA
catcactgccacccagaagactg

SEQ ID NO: 10 - DNA, 23 bp, synthetic construct
  misc_feature 1..23: note = Mouse GAPDH R primer
  source 1..23: mol_type = other DNA
atgccagtgagcttcccgttcag

SEQ ID NO: 11 - DNA, 21 bp, synthetic construct
  misc_feature 1..21: note = Mouse IKZF1 F primer
  source 1..21: mol_type = other DNA
agacaagtgcctgtcagacat

SEQ ID NO: 12 - DNA, 23 bp, synthetic construct
  misc_feature 1..23: note = Mouse IKZF1 R primer
  source 1..23: mol_type = other DNA
ccaggtagttgatggcattgttg

SEQ ID NO: 13 - DNA, 20 bp, synthetic construct
  misc_feature 1..20: note = Mouse IKZF3 F primer
  source 1..20: mol_type = other DNA
gcccagacgctctgaatgac

SEQ ID NO: 14 - DNA, 22 bp, synthetic construct
  misc_feature 1..22: note = Mouse IKZF3 R primer
  source 1..22: mol_type = other DNA
tctcttgcatagctgtaaggca

SEQ ID NO: 15 - DNA, 22 bp, synthetic construct
  source 1..22: mol_type = other DNA
ggaacagcagtggcaagatctc

SEQ ID NO: 16 - DNA, 22 bp, synthetic construct
  source 1..22: mol_type = other DNA
gcaaggctgtagttggtgctca

SEQ ID NO: 17 - DNA, 22 bp, synthetic construct
  source 1..22: mol_type = other DNA
aggtcttcgacaccagccagtt

SEQ ID NO: 18 - DNA, 22 bp, synthetic construct
  source 1..22: mol_type = other DNA
gcacgagaatgagtttggagcg

SEQ ID NO: 19 - DNA, 21 bp, synthetic construct
  source 1..21: mol_type = other DNA
tcctcagcagaacaggaacag

SEQ ID NO: 20 - DNA, 23 bp, synthetic construct
  source 1..23: mol_type = other DNA
ggctcaaaagatgtctcacttgg

SEQ ID NO: 21 - DNA, 20 bp, synthetic construct
  source 1..20: mol_type = other DNA
cggggctgatctgggaaaat

SEQ ID NO: 22 - DNA, 20 bp, synthetic construct
  source 1..20: mol_type = other DNA
cacagcgtaacctcgtcttc

SEQ ID NO: 23 - DNA, 2668 bp, Homo sapiens
  source 1..2668: mol_type = genomic DNA
gcctggagcgcggcagcaagcgtgggaacgcgggcggcgagacggcggcaggacggcggcaggatgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtctaagtgcgtcgcttgggcgccccaccccgtctgcgtcctgcatccatctccctgttacagtggcccgcatcatgattaaagaatgtggatccctctgtctggggtgggatgccttactttgcacttaatttaataagggcattctcggaggagtagacgtttaatacgaagtggcggcatagccctgccgagatgtcggtgatggcctggatgctgtaaccacaacctgtggctaaaaattttattttctatcctttacccgtcattatcattagttgctatgattctttctgcattttcggttaactatcatttccaaagacttgtcattcagtaatattagcagatagctgcttcgataaaggaatttggagtttaaaaatcaacttgtgaaaacaaggttgtttttgtctttatcgtttgttagagttatagatttatgatttcataggcttgattctatgtgaaatatctttttacttttatgcattttaataagatttaaaaatatttagattaaagccccctttaatgagtacaagaaaaactcttggcttgttagaagaaagtatattctttctagaatttggtgcaggaatatgtgttcatatccaggcaaacgggtgtgtttttatcttcagacaatgaaaccttctcctctggggctttgttgccaggaagattagaactaaatttatttttttcatttctgtcatgaaatcattccagatacctcttttcttctttccaaatggttttcacatgtgtttgaaatatttgtacttcgaattgtcggattttccatgtcctcctttctcctttgtgcccagcctgagtcagcaccaatcccgcattcagaacctcccagtgaaagggcagccttcattttgagaaggtggaaggtgttagggtttgggagacagctcatccaatctcccaagtctcatggtggatttgtgactgtgagagtttccggtttaaaatctgaaaagccagatatgcctgtttccttttcccagcaccatgcctgtggaggggacagtcagacccagaggtcctttacgtgtggatggagttcacaggcgaatagaggagaggaccaggggacgtggcttgtcccttttgtccaacaaagcattatatttttaagaatggcagacctgtttgctgaagtgttcataagataacaataggcttgaatctccaattcaaatgaatgtcaaagcacatatctttaatatgctgaatgaatatttatttttgtatccattaaaacagtatattgatctcttttattctttattaaaataaaatgctcttttttaaa

SEQ ID NO: 24 - DNA, 1656 bp, Homo sapiens
  source 1..1656: mol_type = genomic DNA
agacgctggtgcgcctgcttggggctcctgtgctcagctcagcctgagcttccacactcagcgctcagcaatggcccgggggcggggcgcggtcctcgcagattctcaaaggtagccgggatcctcgtccagcagtgtcagctcaggctcagcctccccagagacaacaccgggagcctcatctctctcctcaccctgctgtgactccaccacaggtttctagagccatctgggctttccgggaacctggaccagactctggcccagtaggatgtccccgtgtcctccccagcagagcaggaacagggtgatacagctgtccacttcagagctaggagagatggaactgacttggcaggagatcatgtccatcaccgagctgcagggtctgaatgctccaagtgagccatcatttgagccccaagccccagctccataccttggacctccaccacccacaacttactgcccctgctcaatccacccagattctggcttcccacttcctccaccaccttatgagctcccagcatccacatcccatgtcccagatcccccatactcctatggcaacatggccataccagtctccaagccactgagcctctcaggcctgctcagtgagccgctccaagaccccttagccctcctggacattgggctgccagcagggccacctaagccccaagaagacccagaatccgactcaggattatccctcaactatagcgatgctgaatctcttgagctggaggggacagaggctggtcggcggcgcagcgaatatgtagagatgtacccagtggagtacccctactcactcatgcccaactccttggcccactccaactataccttgccagctgctgagacccccttggccttagagccctcctcaggccctgtgcgggctaagcccactgcacggggggaggcagggagtcgggatgaacgtcgggccttggccatgaagattccttttcctacggacaagattgtcaacttgccggtagatgactttaatgagctattggcaaggtacccgctgacagagagccagctagcgctagtccgggacatccgacgacggggcaaaaacaaggtggcagcccagaactgccgcaagaggaagctggaaaccattgtgcagctggagcgggagctggagcggctgaccaatgaacgggagcggcttctcagggcccgcggggaggcagaccggaccctggaggtcatgcgccaacagctgacagagctgtaccgtgacattttccagcaccttcgggatgaatcaggcaacagctactctcctgaagagtacgcgctgcaacaggctgccgatgggaccatcttccttgtgccccgggggaccaagatggaggccacagactgagctggcccagaggggtggaactgctgatgggatttccttcattcccttctgataaaggtactccccaaccctgagtcccagaaggagctgagttctctagaccagaagaggatgacaatggcaacaagtgtttggaagttccaaggtgtgttcaaagaggcttgccttgagggagggctggaatctgtcttccctgactcggctcctcaggtctttagcctccaccttgtctaagctttggtctataaagtgcgctacagaaa

SEQ ID NO: 25 - DNA, 21 bp, synthetic construct
  source 1..21: mol_type = other DNA
gttacgaatgcttgatgcctc

SEQ ID NO: 26 - DNA, 21 bp, synthetic construct
  source 1..21: mol_type = other DNA
gaggcatcaagcattcgtaac

SEQ ID NO: 27 - DNA, 21 bp, synthetic construct
  source 1..21: mol_type = other DNA
gcaaggcagctcggctttgtc

SEQ ID NO: 28 - DNA, 21 bp, synthetic construct
  source 1..21: mol_type = other DNA
gacaaagccgagctgccttgc

SEQ ID NO: 29 - DNA, 21 bp, synthetic construct
  source 1..21: mol_type = other DNA
tgctgctgtgttgctatcttg

SEQ ID NO: 30 - DNA, 20 bp, synthetic construct
  source 1..20: mol_type = other DNA
acattttgctccttcagccc

(19) United States
(12) Patent Application Publication
(10) Pub. No.: US 2025/0051851 A1
(43) Pub. Date: Feb. 13, 2025

(54) NOVEL BIOMARKER FOR DETECTION OF CANCER METASTASIS

(71) Applicant: INDUSTRY-ACADEMIC COOPERATION FOUNDATION, YONSEI UNIVERSITY, Seoul (KR)

(72) Inventors: Heon Yung GEE, Seoul (KR); Hyun Woo PARK, Seoul (KR); Jongwook OH, Seoul (KR); Yujin SUB, Seoul (KR)

(21) Appl. No.: 18/717,690

(22) PCT Filed: Dec. 8, 2022
(86) PCT No.: PCT/KR2022/019900

§ 371 Date: Jun. 7, 2024

(30) Foreign Application Priority Data
Dec. 9, 2021 (KR) 10-2021-0175471

(51) Int. Cl. C12Q 1/6886; G01N 33/574

(52) U.S. Cl. CPC C12Q 1/6886; G01N 33/5743; C12Q 2600/112; C12Q 2600/136; C12Q 2600/158; G01N 2500/00

(57) ABSTRACT

The present invention relates to the discovery of an agent for measuring the expression level of a protein that induces the acquisition of metastatic ability by tumor cells, or a gene encoding the same, and a method for preventing or treating metastatic cancer by controlling the expression of the protein or gene. The present invention provides effective biomarkers for metastatic cancer that are capable of significantly improving the patient's survival rate when detected early, and provides a method capable of predicting with high reliability the likelihood that primary cancer will progress to metastatic cancer. Thus, the present invention may be useful for efficient prevention or treatment of metastatic cancer by inhibiting the expression of these factors.

### CROSS-REFERENCE TO RELATED APPLICATIONS

This application is a U.S. National Stage Entry of International Patent Application no. PCT/KR2022/019900, filed Dec. 8, 2022, which claims the benefit of priority of Korean Patent Application no. 10-2021-0175471, filed Dec. 9, 2021.

### REFERENCE TO AN ELECTRONIC SEQUENCE LISTING

This application contains a Sequence Listing which has been submitted electronically and is hereby incorporated by reference in its entirety. The Sequence Listing was created on May 24, 2024, is named “24-0717-WO-US_SequenceListing_ST26” and is 39,436 bytes in size.

### TECHNICAL FIELD

The present invention relates to a method of either predicting metastasis by measuring the expression level of a factor that determines whether tumor cells have acquired metastatic ability, or suppressing cancer metastasis by controlling the expression of the factor.

### BACKGROUND ART

Cancer metastasis refers to a phenomenon in which tumor cells detach from the primary tumor tissue, invade the surrounding blood vessels or lymphatic vessels, and migrate to other distal parts of the body through these vessels as passages, forming a new tumor. Since more than 90% of cancer patient deaths are attributable to metastasis from the primary tumor (Nature Reviews Cancer, 2006, 6:49-458), early detection of metastasis in the diagnosis and treatment of patients with primary tumors, or furthermore, prediction of metastasis at a stage before metastasis is very important in reducing the mortality of cancer patients.

In the process in which tumor cells detach from the primary tumor and metastasize through the blood, the transition of primary tumor cells, which are adherent cells, to suspension cells, which are circulating tumor cells (CTCs), is necessary. According to this, a factor involved in the transition from adherent cells to suspension cells may be involved in tumor metastasis.

Specifically, melanoma, a type of skin cancer, is previously known to be a cancer that usually occurs in Western people, but melanoma is a clinically highly important cancer whose incidence has recently increased every year even in Korea. Melanoma is a carcinoma that can be cured by surgical therapy if detected early. However, most malignant melanomas are not accompanied by subjective symptoms such as itching or pain, and appear as ordinary black or blue-black spots, and thus they are very difficult to identify, and even when detected, they are often already in a considerably advanced state. Therefore, factors involved in the transition of tumor cells from adherent cells to suspension cells may be used as biomarkers to predict the progress of metastasis, and are essential for the cure of patients with primary tumors. However, to date, there are no effective biomarkers reported as factors specific to melanoma.

Accordingly, the present inventors have conducted studies on the relationship between the overexpression of specific genes found in metastatic cancer patients and the presence or absence of metastasis in order to discover efficient biomarkers that can detect early or predict whether primary tumors have acquired metastatic ability, and have sought to confirm that the expression of these specific genes plays an important role in the acquisition of metastatic ability by tumor cells and has the potential to serve as a biomarker for cancer metastasis.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### DISCLOSURE

### Technical Problem

The present inventors have made extensive research efforts to discover biomarkers for the efficient detection of metastasis, which constitutes the majority of cancer patient deaths, and to develop a new diagnostic method that can significantly reduce the rate of deaths due to metastatic cancer, by predicting whether the patient's tumor cells will progress to the metastatic stage, and ultimately detecting metastatic cancer at an early stage. As a result, the present inventors have found that IKZF1 and IKZF3 genes are highly expressed specifically when primary tumor cells, which are adherent cells, transition to circulating tumor cells, which are suspension cells, and it is possible to predict and diagnose the acquisition of metastatic ability by tumor cells with high reliability at an early stage before the progression of metastasis by measuring the expression levels of these factors that reprogram anchorage dependency, thereby completing the present invention.

Therefore, an object of the present invention is to provide a diagnostic composition capable of predicting whether cancer will metastasize.

Another object of the present invention is to provide a method of providing information necessary for diagnosis, which is capable of predicting whether cancer will metastasize.

Still another object of the present invention is to provide a composition for preventing or treating metastatic cancer.

Another object of the present invention is to provide a method for screening a composition for preventing or treating metastatic cancer.

Other objects and advantages of the present invention will become more apparent from the following detailed description, the appended claims, and the accompanying drawings.

### Technical Solution

According to one aspect of the present invention, the present invention provides a composition for diagnosing metastatic cancer comprising, as an active ingredient, an agent for measuring the expression level of at least one protein selected from the group consisting of IKZF1 (Ikaros Transcription Factor 1), IKZF3 (Ikaros Transcription Factor 3), NFE2 (Nuclear Factor, Erythroid 2), and IRF8 (Interferon Regulatory Factor 8), or a gene encoding the same.

The present inventors have made extensive research efforts to discover biomarkers for the efficient detection of metastasis, which constitutes the majority of cancer patient deaths, and to develop a new diagnostic method that can significantly reduce the rate of deaths due to metastatic tumors, by predicting whether the patient's tumor cells will progress to the metastatic stage, and ultimately detecting metastatic cancer at an early stage. As a result, the present inventors have found that IKZF1 and IKZF3 genes are highly expressed specifically when primary tumor cells, which are adherent cells, transition to circulating tumor cells, which are suspension cells, and it is possible to predict and diagnose the acquisition of metastatic ability by tumor cells with high reliability at an early stage before the progression of metastasis by measuring the expression levels of these factors that reprogram anchorage dependency.

In the present specification, the term “metastatic cancer” refers to a new tumor formed when tumor cells detached from the primary tumor tissue invade the surrounding blood vessels or lymphatic vessels and migrate to other distal parts of the body through these vessels as passages. Since more than 90% of cancer patient deaths are attributable to metastasis from the primary tumor (Nature Reviews Cancer, 2006, 6:449-458), suppressing metastasis to reduce the mortality of cancer patients is as important as treating primary cancer.

The mechanism by which tumor cells acquire mobility during the metastatic process is explained by an epithelial-to-mesenchymal transition (EMT), in which tumor epithelial cells acquire the characteristics of mesenchymal cells through genetic mutation, and a mesenchymal-to-epithelial transition (MET), which is the opposite process (J Clin Invest. 2009, 119:1417-1419). In other words, epithelial cells that have the characteristics of mesenchymal cells detach from their original location due to their weak intercellular adhesion and migrate to blood vessels, and the cells that have been migrating through blood vessels regain their original epithelial characteristics (MET) and settle at secondary sites distal to the primary site, causing a tumor to form.

The present inventors have found that IKZF1, IKZF3, NFE2 or IRF8 gene is highly expressed specifically during the transition from adherent tumor cells to suspension cells, and the expression levels of these genes are significantly high even in circulating tumor cells (CTCs), and thus have found that IKZF1, KZF3, NFE2, or IRF8 can serve as a highly reliable biomarker in the process in which cancer cells in the primary tissue acquire the suspension cell phenotype suitable for metastasis and metastasize to secondary sites. Therefore, it can be determined that, when IKZF1, KZF3, NFE2, or IRF8 is highly expressed at the primary tumor site, circulating tumor cells (CTCs) that cause metastatic cancer have already been generated or are at a high risk of being generated in the future. Accordingly, “diagnosis of metastatic cancer” is used in the same sense as “diagnosis of cancer metastasis”, “prediction of epithelial-to-mesenchymal transition”, “prediction of generation of circulating tumor cells”, or “prediction of cancer prognosis”.

In the present specification, the term “diagnosis” or “diagnosing” includes determination of the susceptibility of a subject to a particular disease, determination of whether a subject currently has a particular disease, and determination of the prognosis of a subject suffering from a particular disease.

In the present specification, the term “composition for diagnosing” refers to an integrated mixture or device including a means for detecting the expression level of the IKZF1, IKZF3, NFE2 or IRF8 protein or the IKZF1, IKZF3, NFE2 or IRF8 gene in order to determine whether tumor cells in a subject have acquired metastatic ability or are likely to acquire metastatic ability. Therefore, the term may also be expressed as a “kit for diagnosing”.

According to a specific embodiment of the present invention, the agent for measuring the expression level of a gene encoding at least one protein selected from the group consisting of IKZF1, IKZF3, NFE2, and IRF8 is a primer or probe that binds specifically to the nucleic acid molecule of the gene.

In the present specification, the term “nucleic acid molecule” is meant to encompass DNA (gDNA and cDNA) and RNA molecules. Nucleotides, which are the basic structural units in nucleic acid molecules, include not only natural nucleotides, but also analogues having modified sugar or base moieties (Scheit, Nucleotide Analogs, John Wiley, New York (1980); Uhlman and Peyman, Chemical Reviews, 90:543-584 (1990)).

As used in the present specification, the term “primer” refers to an oligonucleotide which serves as a starting point for synthesis under conditions in which the synthesis of a primer extension product complementary to a nucleic acid chain (template) is induced, i.e., the presence of nucleotides and a polymerase such as DNA polymerase, and suitable temperature and pH. Specifically, the primer is a deoxyribonucleotide single chain. The primers that are used in the present invention may include naturally occurring dNMPs (i.e., dAMP, dGMP, dCMP and dTMP), modified nucleotides or non-natural nucleotides. In addition, the primers may also include ribonucleotides.

The primer of the present invention may be an extension primer which is annealed to a target nucleic acid and forms a sequence complementary to the target nucleic acid by a template-dependent nucleic acid polymerase. The extension primer is extended to the location where an immobilized probe is annealed and occupies the area at which the probe is annealed.

The extension primer that is used in the present invention includes a hybridizing nucleotide sequence complementary to a specific nucleotide sequence of a target nucleic acid, e.g., the IKZF1, IKZF3, NFE2 or IRF8 gene. The term “complementary” means that a primer or a probe is complementary enough to selectively hybridize to a target nucleic acid sequence under a certain annealing or hybridization condition. The term “complementary” encompasses both “substantially complementary” and “perfectly complementary”, and specifically, it means perfectly complementary. In the present specification, the term “substantially complementary sequence” is meant to include not only a completely matching sequence, but also a sequence partially mismatching with the sequence to be compared within a range where it can anneal to a particular sequence and serve as a primer.

The primer should be long enough to prime the synthesis of an extension product in the presence of a polymerase. The suitable length of the primer is determined depending on a number of factors, such as temperature, pH and the source of the primer, but is typically 15 to 30 nucleotides. Short primer molecules generally require lower temperatures to form a sufficiently stable hybrid complex with a template. The design of such a primer can be easily carried out by those skilled in the art with reference to a target nucleotide sequence using, for example, a program for primer design (e.g., PRIMER 3 program).

In the present specification, the term “probe” refers to a natural or modified monomer or a linear oligomer having linkages, which includes a deoxyribonucleotide and ribonucleotide that can be hybridized with a specific nucleotide sequence. Specifically, the probe is single-stranded for maximum efficiency in hybridization. More specifically, the probe is a deoxyribonucleotide. As the probe that is used in the present invention, a sequence perfectly complementary to a specific nucleotide sequence of the IKZF1, IKZF3, NFE2 or IRF8 gene may be used, but a sequence substantially complementary thereto may also be used as long as specific hybridization is not interrupted. In general, the stability of a duplex formed by hybridization tends to be determined by the match of terminal sequences, and thus it is preferred to use a probe which is complementary to the 3′-end or 5′-end of the target sequence.

Conditions suitable for hybridization may be determined with reference to the contents disclosed in Joseph Sambrook, et al., Molecular Cloning, A Laboratory Manual, Cold Spring Harbor Laboratory Press, N.Y. (2001), and Haymes, B. D., et al., Nucleic Acid Hybridization, A Practical Approach, IRL Press, Washington, D.C. (1985).

According to a specific embodiment of the present invention, the agent for measuring the expression level of at least one protein selected from the group consisting of IKZF1, IKZF3, NFE2, and IRF8 is an antibody or an antigen-binding fragment thereof that binds specifically to the at least one protein selected from the group consisting of IKZF1, IKZF3, NFE2, and IRF8, or an aptamer that binds specifically to at least one protein selected from the group consisting of IKZF1, IKZF3, NFE2, and IRF8.

According to the present invention, the IZFK1, IKZF3, NFE2 or IRF8 protein of the present invention may be detected according to an immunoassay method using an antigen-antibody reaction and used to analyze whether tumor cells have acquired metastatic ability. This immunoassay may be performed according to various immunoassay or immunostaining protocols which have been conventionally developed.

For example, when the method of the present invention is performed according to a radioimmunoassay method, antibodies labeled with radioisotopes (e.g., C¹⁴, I¹²⁵, P³², and S³⁵) may be used. In the present invention, the antibody that specifically recognizes the IZFK1 or IKZF3 protein is a polyclonal or monoclonal antibody, preferably a monoclonal antibody.

The antibody of the present invention may be produced by methods commonly practiced in the art, for example, the fusion method (Kohler and Milstein, European Journal of Immunology, 6:511-519 (1976)), the recombinant DNA method (U.S. Pat. No. 4,816,567), or the phage antibody library method (Clackson et al, Nature, 352:624-628 (1991), and Marks et al, J. Mol. Biol., 222:58, 1-597 (1991)). General procedures for antibody production are described in detail in Harlow, E. and Lane, D., Using Antibodies: A Laboratory Manual, Cold Spring Harbor Press, New York, 1999; and Zola, H., Monoclonal Antibodies: A Manual of Techniques, CRC Press, Inc., Boca Raton, Florida, 1984.

By analyzing the intensity of the final signal by the above-described immunoassay process, it is possible to predict whether a tumor has metastasized or is likely to metastasize. In other words, if the signal for the IZFK1, IKZF3, NFE2 or IRF8 protein in a sample from a subject is stronger than that in a normal sample, it is determined that the tumor in the subject has metastasized or is highly likely to metastasize in the future.

In the present specification, the term “antigen-binding fragment” refers to a portion of a polypeptide, to which an antigen can bind, among the entire structure of an immunoglobulin. Examples of the antigen-binding fragment include, but are not limited to, F(ab′)2, Fab′, Fab, Fv, and scFv.

In the present specification, the term “specifically binding” has the same meaning as “specifically recognizing”, and means that an antigen and an antibody (or a fragment thereof) specifically interact with each other by an immunological reaction.

In the present invention, an aptamer that specifically binds to the IZFK1, IKZF3, NFE2 or IRF8 protein may be used instead of an antibody. In the present specification, the term “aptamer” refers to a single-stranded nucleic acid (RNA or DNA) molecule or peptide molecule that binds to a specific target substance with high affinity and specificity. General contents regarding aptamers are disclosed in detail in Hoppe-Seyler F, Butz K “Peptide aptamers: powerful new tools for molecular medicine”. J Mol Med. 78 (8): 426-30 (2000); Cohen B A, Colas P, Brent R. “An artificial cell-cycle inhibitor isolated from a combinatorial library”. Proc Natl Acad Sci USA. 95 (24): 14272-7 (1998).

According to a specific embodiment of the present invention, the cancer that can be diagnosed by the composition of the present invention is skin cancer.

More specifically, the skin cancer is melanoma.

According to another aspect of the present invention, the present invention provides a method of providing information necessary for diagnosis of metastatic cancer, comprising a step of measuring the expression level of at least one protein selected from the group consisting of IKZF1, IKZF3, NFE2, and IRF8, or a gene encoding the same, in a biological sample isolated from a subject.

Since the IZFK1, IKZF3, NFE2 or IRF8 protein of the present invention, the gene encoding the same, and metastatic cancer that can be diagnosed using the same have already been described in detail, the description thereof will omitted to avoid excessive overlapping.

The present inventors have discovered for the first time that the IZFK1, IKZF3, NFE2 or IRF8 protein and the acquisition of metastatic ability by tumor cells have a positive correlation. Accordingly, it is determined that, when the IZFK1, IKZF3, NFE2 or IRF8 protein or the gene encoding the same is highly expressed in a subject, the subject has tumor cells with metastatic ability or will have the tumor cells in the future.

In the present specification, the term “highly expressed” means that the expression level of the protein or gene is significantly higher than that in a control group in which metastasis has not occurred or the likelihood of metastasis is low. Specifically, the term means that the expression level is 130% or more, more specifically 150% or more, most specifically 170% or more, of that in the control.

In the present specification, the term “individual” refers to a subject that provides a sample in which the expression level of the IZFK1, IKZF3, NFE2 or IRF8 protein or the gene encoding the same is to be measured, and which is ultimately to be analyzed as to the acquisition of metastatic ability by tumor cells. The term “subject” includes, without limitation, humans, mice, rats, guinea pigs, dogs, cats, horses, cows, pigs, monkeys, chimpanzees, baboons or rhesus monkeys. Specifically, the subject is a human. Because the composition of the present invention provides information for predicting not only information on whether tumor cells have acquired metastatic ability, but also information for predicting whether tumor cells have a genetic risk of acquiring metastatic ability in the future, the subject of the present invention may be a metastatic cancer patient and may also be a patient with primary cancer that has not yet acquired metastatic ability.

According to another aspect of the present invention, the present invention provides a composition for preventing or treating metastatic cancer comprising, as an active ingredient, an inhibitor of at least one selected from the group consisting of IKZF1, IKZF3, NFE2, and IRF8.

According to the present invention, the present inventors have discovered genes that are expressed only in suspension cells but not in adherent cells, and found that cells in which these genes had been artificially overexpressed transition to suspension cells, unlike their original phenotype. Furthermore, it has been experimentally proven that inhibiting the expression of these genes in tumor cells can suppress tumor metastasis by inhibiting the formation of CTCs.

In the present specification, the term “inhibitor” refers to a substance that reduces the activity or expression of a target gene so that the activity or expression of the target gene becomes undetectable or is at an insignificant level or the biological function of the target gene may be significantly reduced.

Examples of the inhibitor of the target gene include, but are not limited to, shRNA, siRNA, miRNA, ribozyme, peptide nucleic acid (PNA), and an antisense oligonucleotide, which inhibit the expression of the gene whose sequence is already known in the art at the gene level; a CRISPR system comprising a guide RNA that recognizes the target gene; antibodies or aptamers that inhibit the expression at the protein level; and compounds, peptides and natural products that inhibit the activity of the target gene. In addition, any means for gene and protein level inhibition known in the art may be used.

In the present specification, the term “small hairpin RNA (shRNA)” refers to a single-stranded RNA sequence consisting of 50 to 70 nucleotides, which forms a stem-loop structure in vivo and creates a tight hairpin structure for inhibiting target gene expression by RNA interference. Usually, a double-stranded stem is formed by complementary base pairing of a long RNA consisting of 19 to 29 nucleotides on both sides of the loop consisting of 5 to 10 nucleotides, and for constitutive expression, the double-stranded stem is transduced into cells via a vector including U6 promoter and is usually delivered to daughter cells so that inhibition of expression of the target gene is inherited.

In the present specification, the term “siRNA” refers to a short double-stranded RNA capable of inducing RNA interference (RNAi) through cleavage of a specific mRNA. The siRNA is composed of a sense RNA strand having a sequence homologous to the mRNA of the target gene and an antisense RNA strand having a sequence complementary thereto. The total length thereof may be 10 to 100 bases, preferably 15 to 80 bases, most preferably 20 to 70 bases, and the ends thereof may be blunt or cohesive as long as the siRNA is capable of inhibiting the expression of the target gene by the RNAi effect. The cohesive ends may have a 3′ end overhang and a 5′ end overhang.

In the present specification, the term “microRNA (miRNA)” refers to an oligonucleotide that is not expressed in cells, and means a single-stranded RNA molecule that inhibits target gene expression by complementary binding to the mRNA of the target gene while having a short stem-loop structure.

In the present specification, the term “ribozyme” refers to a kind of RNA which is an RNA molecule having the same function as an enzyme that recognizes and cleaves a specific RNA nucleotide sequence by itself. The ribozyme is a nucleotide sequence complementary to a target mRNA strand and is composed of a region that binds specifically to the target mRNA and a region that cleaves the target RNA.

In the present specification, the term “peptide nucleic acid (PNA)” refers to a molecule capable of complementary binding to DNA or RNA while having both nucleic acid and protein properties. PNA is not found in nature, is artificially synthesized by a chemical method, and regulates target gene expression by forming a double strand through hybridization with a natural nucleic acid having a nucleotide sequence complementary thereto.

In the present specification, the term “antisense oligonucleotide” refers to a nucleotide sequence complementary to a specific mRNA sequence. Specifically, it refers to a nucleic acid molecule which binds to a complementary sequence in a target mRNA and inhibits the activities of the target mRNA, which are essential for translation into proteins, translocation into cytoplasm, maturation, or all other overall biological functions. The antisense oligonucleotide may be modified at one or more bases, sugars or backbone positions to enhance efficacy thereof (De Mesmaeker et al., Curr Opin Struct Biol., 5 (3): 343-55, 1995). The oligonucleotide backbone may be modified with phosphorothioate, phosphotriester, methylphosphonate, short-chain alkyl, cycloalkyl, short-chain heteroatomic, heterocyclic sugar sulfonate, or the like.

In the present specification, the term “guide RNA (gRNA)” refers to an RNA molecule used in a gene editing system that recognizes a target gene and induces a nuclease to specifically cleave the recognized site. Typical examples of this gene editing system include a CRISPR (Clustered Regularly Interspaced Short Palindromic Repeats) system.

According to the present invention, the expression inhibitor of the present invention may be a specific antibody that inhibits the activity of the protein encoded by the gene. An antibody that specifically recognizes the target protein is a polyclonal or monoclonal antibody, preferably a monoclonal antibody.

Since the antibodies or aptamers that may be used in the present invention have already been described in detail, description thereof will be omitted to avoid excessive overlapping.

In the present specification, the term “prevention” or “preventing” means inhibiting the occurrence of a disorder or disease in a subject who has never been diagnosed as having the disorder or disease, but is likely to suffer from such disorder or disease.

In the present specification, the term “treatment” or “treating” means (a) inhibiting the progress of a disorder, disease or symptom; (b) alleviating the disorder, disease or symptom; or (c) eliminating the disorder, disease or symptom. When the composition of the present invention is administered to a subject, it functions to inhibit the generation of circulating tumor cells while inhibiting the expression of the IZFK1, IKZF3, NFE2 or IRF8 protein or the gene encoding the same, thereby inhibiting, eliminating, or alleviating the progress of symptoms resulting from a tumor, specifically a metastatic tumor. Thus, the composition of the present invention may serve as a therapeutic composition for these diseases by itself, or may be administered in combination with other pharmacological ingredients and applied as a therapeutic aid for the diseases. Accordingly, as herein used, the term “treatment” or “therapeutic agent” is meant to encompass “treatment aid” or “therapeutic aid agent”.

According to a specific embodiment of the present invention, the cancer that can be prevented or treated by the composition of the present invention is skin cancer.

More specifically, the skin cancer is melanoma.

According to another aspect of the present invention, the present invention provides a method for screening a composition for preventing or treating metastatic cancer, comprising steps of:
(a) bringing a test substance into contact with a biological sample containing at least one protein selected from the group consisting of IKZF1, IKZF3, NFE2 and IRF8, a gene encoding the protein, or cells expressing the protein or gene; and
(b) measuring the expression level of the at least one protein selected from the group consisting of IKZF1, IKZF3, NFE2 and IRF8, or the gene encoding the same, in the biological sample.

When the expression level of the protein or the gene in the biological sample decreases, the test substance is determined to be a composition for preventing or treating metastatic cancer.

According to a specific embodiment of the present invention, the biological sample contains tumor tissue or tumor cells.

Since the anchorage dependency-reprogramming factors that are used in the present invention and the type of cancer that can be prevented or treated by controlling the expression of these factors have already been described in detail, the description thereof will be omitted to avoid excessive overlapping.

In the present invention, the term “biological sample” refers to any sample containing cells expressing the above-described gene, which is obtained from mammals, including humans. Examples of the biological sample include, but are not limited to, tissues, organs, cells, or cell cultures. More specifically, the biological sample may be tumor tissue, tumor cells, or a culture thereof.

The term “test substance” as used while referring to the screening method of the present invention refers to an unknown substance which is used in screening to examine whether it affects the activity or expression level of the gene of the present invention by being added to a sample containing cells expressing the gene. Examples of the test substance include, but are not limited to, compounds, nucleotides, peptides, and natural extracts. The step of measuring the expression level or activity of the gene in the biological sample treated with the test substance may be performed by various expression level and activity measurement methods known in the art.

According to another aspect of the present invention, the present invention provides a method for diagnosing cancer metastasis comprising a step of administering, to a subject, a composition for diagnosing metastatic cancer comprising, as an active ingredient, an agent for measuring the expression level of the IKZF1 (Ikaros Transcription Factor 1), IKZF3 (Ikaros Transcription Factor 3), NFE2 (Nuclear Factor, Erythroid 2), or IRF8 (Interferon Regulatory Factor 8) protein, or the gene encoding the same.

According to another aspect of the present invention, the present invention provides a method for preventing or treating metastatic cancer comprising a step of administering, to a subject, a composition for preventing or treating metastatic cancer comprising, as an active ingredient, an inhibitor of IKZF1, IKZF3, NFE2 or IRF8.

In the present invention, the meaning of metastatic cancer, the genes of the present invention, and metastatic cancer that can be diagnosed, prevented, or treated using the genes have already been described in detail, and thus the description thereof will be omitted to avoid excessive overlapping.

### Advantageous Effects

The features and advantages of the present invention are summarized as follows:
(a) The present invention has discovered agents for measuring the expression levels of proteins or genes encoding the same that induce the acquisition of metastatic ability by tumor cells, and provides a method of preventing or treating metastatic cancer by controlling the expression of these proteins or genes.
(b) The present invention provides effective biomarkers for metastatic cancer that are capable of significantly improving the patient's survival rate when detected early, and provides a method capable of predicting with high reliability the likelihood that primary cancer will progress to metastatic cancer. Thus, the present invention may be useful for efficient prevention or treatment of metastatic cancer by inhibiting the expression of these factors.

### BRIEF DESCRIPTION OF DRAWINGS

FIG. 1A shows the results of comparing, by RT-PCR, the IKZF1 and IKZF3 RNA expression levels of adherent and suspension cells relative to control cells in the mouse cell line B16F10 and the human cell lines A375 and SKMEL28. FIG. 1B shows the results of comparing, by RT-PCR, the IKZF1 and IKZF3 RNA expression levels of adherent and suspension cells relative to control cells in the human cell lines IGR1 and G361.

FIG. 2 shows the results of analysis performed using RNA sequencing data of GEO126076 (Cancer research 2019; 79 (10): 2736-2747), and shows the results of RNA sequencing performed according to color after dividing into center, border, and adjacent tissues based on the distance from the primary site. FIG. 2 also shows a volcano plot of IKZF1 and IKZF3 genes in the center and adjacent tissues.

FIG. 3 shows the results of analysis performed using RNA sequencing data of E-MTAB-7621 (Science. 2019; 363 (6427): 644-649), and shows the results of RNA sequencing performed according to color after dividing into center, border, and adjacent tissues based on the distance from the primary site. FIG. 3 also shows a volcano plot of IKZF1 and IKZF3 genes in the center and adjacent tissues.

FIG. 4A shows the expression levels of IKZF1 and IKZF3 genes, obtained by analysis of RNA sequencing data of GEO157743 (Cancer Discov 11 (3): 678-695), compared to a control.

FIG. 4B shows the expression level of each of IKZF1 and IZKZF3 genes, obtained by analysis of RNA sequencing data of GEO157743 (Cancer Discov 11 (3): 678-695), compared to a control.

FIG. 5 is a schematic diagram summarizing a process of performing an AST assay, an experimental method that can show the transition from adherent cells to suspension cells.

FIG. 6A shows the results of comparing, by RT-PCR, the IRF8 and NFE2 RNA expression levels of adherent and suspension cells relative to control cells in the mouse cell line B16F10 and the human cell lines A375 and SKMEL28. FIG. 6B shows the results of comparing, by RT-PCR, the IRF8 and NFE2 RNA expression levels of adherent and suspension cells relative to control cells in the human cell lines IGR1 and G361.

FIG. 7 shows the results of analysis performed using RNA sequencing data of GEO126076 (Cancer research 2019; 79 (10): 2736-2747), and shows the results of RNA sequencing performed according to color after dividing into center, border, and adjacent tissues based on the distance from the primary site. FIG. 7 also shows a volcano plot of IKZF1, IKZF3, IRF8 and NFE2 genes in the center and adjacent tissues.

FIG. 8 shows the results of analysis performed using RNA sequencing data of GSE52031 (Cell report 2014 8; 7 (3): 645-53), and shows RNA sequencing data for each of a primary site, blood circulating tumor cells (CTCs), and a metastatic site when metastasis occurred.

FIG. 9 shows the results of analyzing RNA sequencing data of GEO157743 (Cancer Discov 11 (3): 678-695), and shows the results of comparing the expression levels of IKZF1, IKZF3, NFE2, and IRF8 with controls.

FIG. 10 shows data indicating the results of performing single-cell RNA sequencing after isolating only GFP+ tumor cells by FACS sorting from a primary site, circulating tumor cells, and a metastatic site (lung), when lung metastasis occurred after injection of B16F10 (mouse melanoma cell line) into the paw pads of mice.

FIG. 11 shows that when the IKZF1 gene in the B16F10 cell line was knocked out (KO) using CRISPR technology, the efficiency of the transition from adherent cells to suspension cells decreased compared to that in the control group.

FIG. 12 shows the results of counting circulating tumor cells after administration of lenalidomide and pomalidomide, which are drugs that induce the degradation of IKZF1 and IKZF3.

FIG. 13 shows fluorescence images of mice obtained using an in vivo optical imaging system after injecting 5×10⁵ B16F10 (GFP⁺) cells into the paw pad of 5 to 6 week-old melanoma mouse models.

### MODE FOR INVENTION

Hereinafter, the present invention will be described in more detail by way of examples. These examples are only for illustrating the present invention in more detail, and it will be apparent to those skilled in the art that the scope of the present invention according to the subject matter of the present invention is not limited by these examples.

### EXAMPLES

### Experimental Methods and Analysis Methods

### Cell Lines

A total of five cell lines were used, one of which was a mouse melanoma cell line and the other four were human melanoma cell lines. B16F10, a mouse melanoma cell line, was purchased from Imanis Life Sciences, and human melanoma cell lines, A375A (ATCC), SK-MEL-28 (ATCC), G361 (ATCC), and IGR1 (AcceGen) were purchased separately and used in experiments. All animal experiments were approved by the Yonsei University Institutional Animal Care and Use Committee (IACUC).

### AST Assay

In an experimental method that can show the process of the transition from adherent cells to suspension cells, cells were plated on about 50% of a cell plate, and when the cell density reached 100%, the culture medium was replaced. Thereafter, the culture medium was not replaced. On day 3, suspension cells were harvested by removing the culture medium.

### Quantitative Real-Time PCR

RNA was extracted using an RNA extraction kit (GeneAll, Korea) from control cells (cell density: 70%), adherent cells (cells attached to the cell plate on day 3), and suspension cells (cells suspended in culture medium on day 3).

cDNA was synthesized from the extracted RNA using a reverse transcription kit (TaKaRa, Japan).

PCR was performed using SYBR Premix (TaKaRA, Japan). GAPDH was used as a housekeeping gene, and the results were expressed relative to the control cells.

### Primers Used in Quantitative Real-Time PCR

The sequences of the primers used in quantitative real-time PCR are as follows.

(1) Human AST gene primers:
GAPDH (forward),
GTC TCC TCT GAC TTC AAC AGC G;
GAPDH (reverse),
ACC ACC CTG TTG CTG TAG CCA A;
IKZF1 (forward),
GCT GCC ACA ACT ACT TGG AAA GC;
IKZF1 (reverse),
AGT CTG TCC AGC ACG AGA GAT C;
IKZF3 (forward)
GCC AAA GAA GAG ATG CGC TCA C;
IKZF3 (reverse)
GGC GTT ATT GAT GGC TTG GTC C
NFE2 (forward),
GGA ACA GCA GTG GCA AGA TCT C;
NFE2 (reverse),
GCA AGG CTG TAG TTG GTG CTC A;
IRF8 (forward),
AGG TCT TCG ACA CCA GCC AGT T;
IRF8 (reverse),
GCA CGA GAA TGA GTT TGG AGC G
(2) Mouse AST gene primers:
GAPDH (forward),
CAT CAC TGC CAC CCA GAA GAC TG;
GAPDH (reverse),
ATG CCA GTG AGC TTC CCG TTC AG;
IKZF1 (forward),
AGA CAA GTG CCT GTC AGA CAT;
IKZF1 (reverse),
CCA GGT AGT TGA TGG CAT TGT TG;
IKZF3 (forward),
GCC CAG ACG CTC TGA ATG AC;
IKZF3 (reverse)
TCT CTT GCA TAG CTG TAA GGC A;
NFE2 (forward),
TCC TCA GCA GAA CAG GAA CAG;
NFE2 (reverse),
GGC TCA AAA GAT GTC TCA CTT GG;
IRF8 forward),
CGG GGC TGA TCT GGG AAA AT;
IRF8 (reverse),
CAC AGC GTA ACC TCG TCT TC

The nucleotide sequences of the IKZF1 and IKZF3 genes are attached to the present specification as SEQ ID NOs: 1 and 2, respectively.

The nucleotide sequences of the IRF8 and NFE2 genes are attached to the present specification as SEQ ID NOs: 23 and 24, respectively.

### RNA Sequencing Analysis

RNA sequencing data of melanoma patient tissue were downloaded from the Gene Expression Omnibus (GEO) archive. As melanoma patient's primary site tissue, GEO126076 was used, and as melanoma patient's vlood circulating tumor cell tissue, GEO157743 was used. Mouse melanoma RNA sequencing data of Science 2019 paper were downloaded from the EMBL-EBI database (E-MTAB-7621). Differential expression analysis was performed using R software and DESeq2 package. Statistically significant DEGs were defined as p-value<0.05 and gene expression fold-change value>1.

### Melanoma Mouse Model

It could be seen that lung metastasis occurred 8 weeks after injecting 5×10⁵ B16F10 (GFP⁺) cells into the paw pads of 5 to 6-week-old mice. At this time, GFP+FACS sorting was performed to obtain only tumor cells from the primary site, blood, and lung site, and then single-cell RNA sequencing analysis was performed.

### In Vitro Experiment Using IKZF1 Knock-Out B16F10 Cell Line

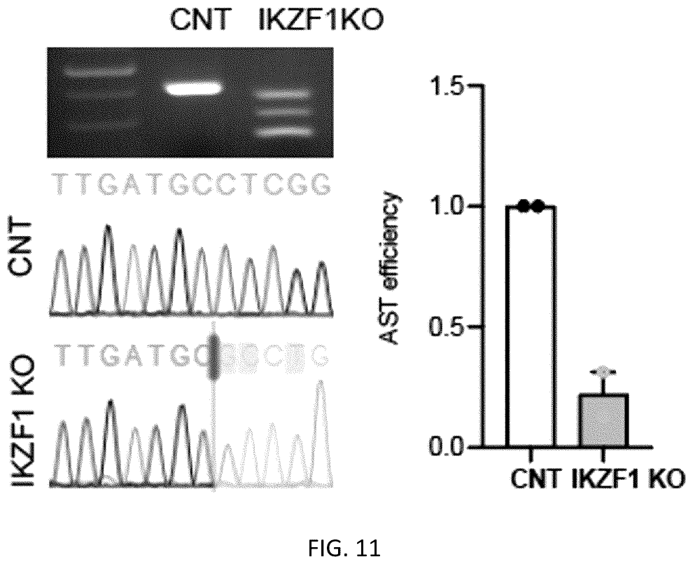

The IKZF1 gene in the B16F10 cell line was knocked out (KO) using CRISPR technology. For the guide RNA used in CRISPR, as a result of observing the sequence and band size using PCR, it could be confirmed that the IKZF1 gene was properly knocked out by CRISPR technology (FIG. 11). AST assay was performed on the IKZF1 KO cells versus control (CNT) cells to measure the efficiency of transition from adherent cells to suspension cells.

The guide RNA sequences used in knock-out (KO) using CRISPR are as follows:

IKZF1 guide 1 (forward):
GTT ACG AAT GCT TGA TGC CTC;
IKZF1 guide 1 (reverse):
GAG GCA TCA AGC ATT CGT AAC;
IKZF1 guide 2 (forward):
GCA AGG CAG CTC GGC TTT GTC;
IKZF1 guide 2 (reverse):
GAC AAA GCC GAG CTG CCT TGC.

In addition, the sequence of the primer used in PCR to confirm knock-out (KO) is as follows:

(Forward):
TGC TGC TGT GTT GCT ATC TTG,
(reverse):
ACA TTT TGC TCC TTC AGC CC

### In Vivo Experiment Using IKZF Inhibitor

The change in the efficiency of transition to circulating tumor cells upon the inhibition of IKZF1 and IKZF3 was observed by injecting lenalidomide or pomalidomide, a substance known to have a mechanism of inducing the degradation of IKZF1 and IKZF3, into a melanoma mouse model, counting circulating tumor cells.

### Experimental Results

Expression Levels of IKZF1 and IKZF3 in Melanoma Cell Lines Increase with Transition to Suspension Cells

While suspension cells are more difficult to culture in vitro than adhesion cells, they are easier to migrate freely, and thus can considered as circulating tumor cells (CTC) that play a key role in the process in which tumor cells detach from the primary tumor tissue and metastasize to distal sites of through the blood. Although genes whose expression is mutually exclusive between adherent cells and suspension cells were not clearly known previously, the present inventors have observed that that among several anchorage dependency-reprogramming factors, IKZF1 and IKZF3, in particular, increase when melanoma cell lines, a type of skin cancer, transition from adherent cells to suspension cells, and thus have found that these genes can be used as markers for the metastatic progression of melanoma, and can also be used as targets for suppressing metastasis.

In order to confirm that IKZF1 and IKZF3 genes are expressed during the transition from adherent cells to suspension cells, suspension cells were collected through AST assay, and the collected sample was analyzed by quantitative real-time PCR and RNA sequencing analysis.

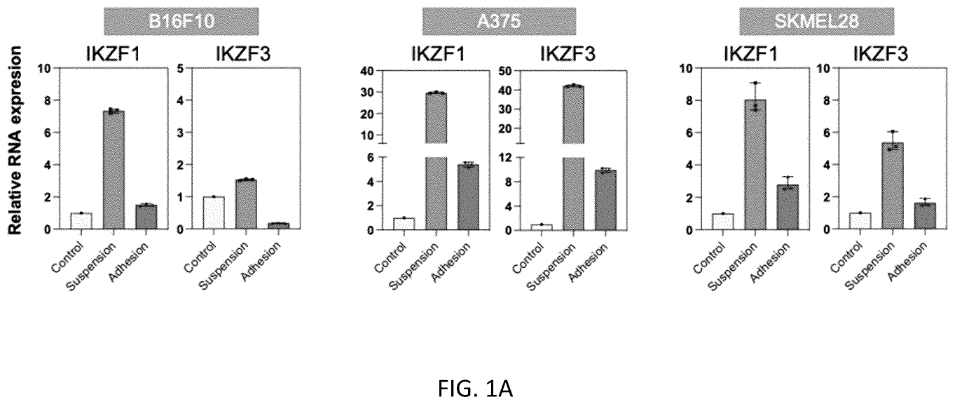

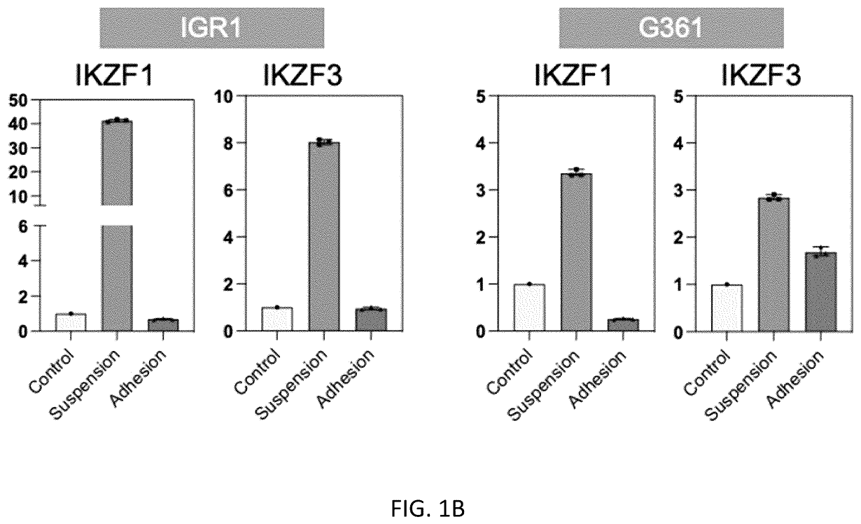

First, it was confirmed that the expression levels of IKZF1 and IKZF3 increased commonly in all of five melanoma cell lines, including one mouse melanoma cell line and four human melanoma cell lines (FIGS. 1A-1B). These results were confirmed by comparing the relative expression levels of IKZF1 and IKZF3 RNA in adhesive and suspension cells with those in control cells through RT-PCR.

### Increased Expression Levels of IKZF1 and IKZF3 are Also Confirmed Through RNA Sequencing Analysis.

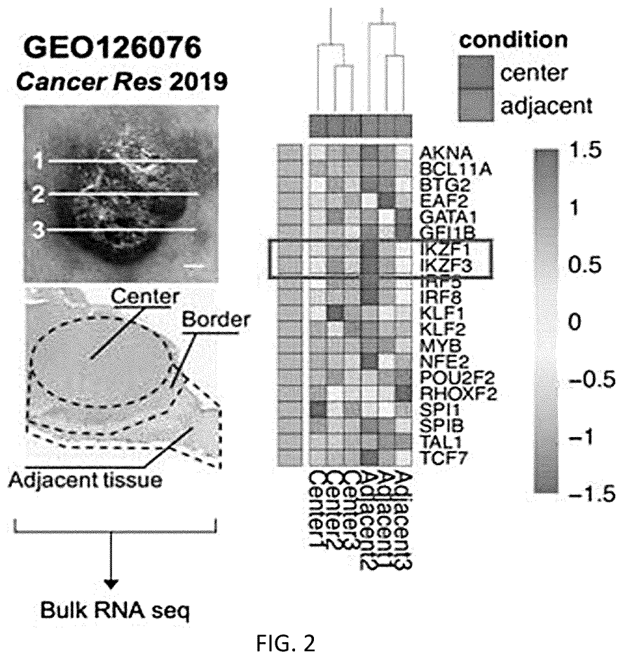

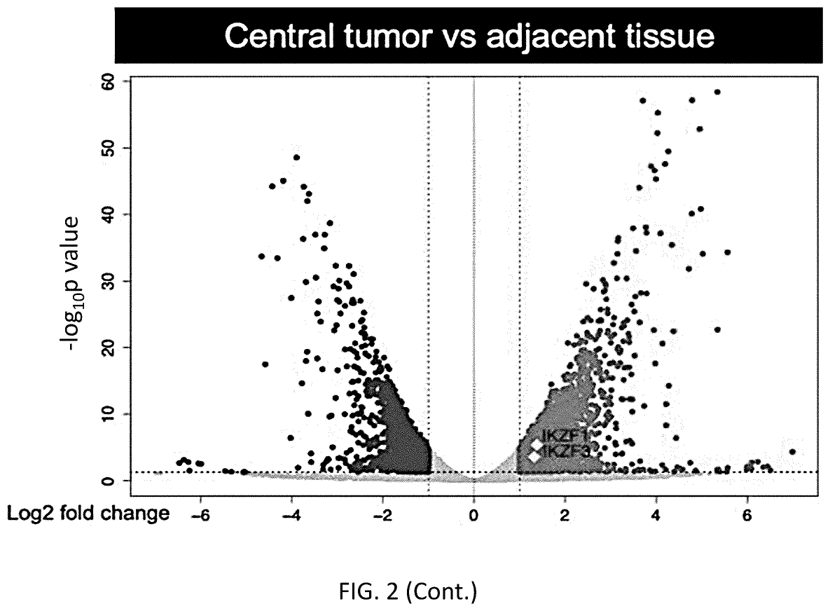

The results of analysis performed using RNA sequencing data from GEO126076 (Cancer research 2019; 79 (10): 2736-2747), a human melanoma patient tissue, also show that the expression levels of IKZF1 and IKZF3 increased when cancer metastasized. These results were confirmed by dividing the melanoma patient's primary site into center, border, and adjacent tissues and then measuring the gene expression levels in each tissue through analysis of RNA sequencing data. It was observed that, in the adjacent tissue far away from the center, which is the primary site, the relative expression levels of IKZF1 and IKZF3 were higher than those in the center tissue (FIG. 2). As a result of visualizing the RNA sequencing data in a volcano plot and presenting the data as log 2 fold-change, it was confirmed that the expression levels in the adjacent tissue were 1.5-fold higher in those in the center tissue, which means a statistically significant increase (FIG. 2).

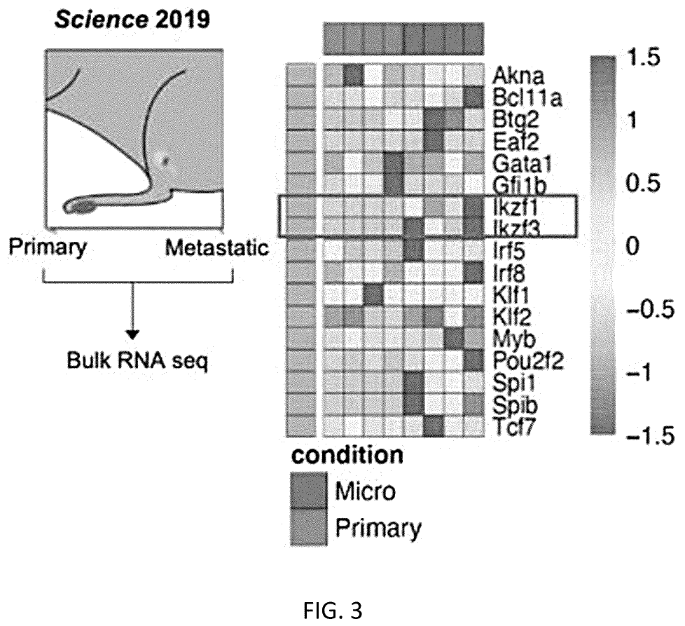

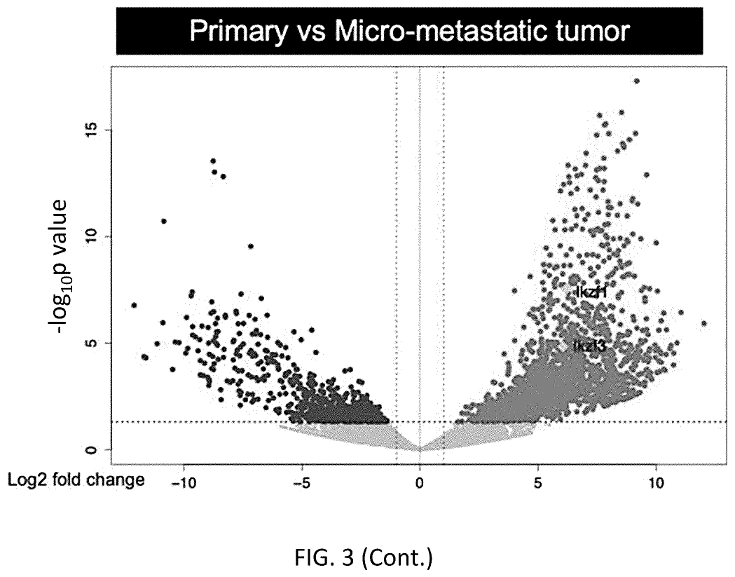

The results of analysis performed using RNA sequencing data from E-MTAB-7621 (Science. 2019; 363 (6427): 644-649), a mouse melanoma model, also showed increased expression of IKZF1 and IKZF3 in the metastatic site. These results were confirmed through the data obtained by RNA sequencing of each of primary and metastatic sites after detaching the lymph node tissue, when metastasis to the lymph nodes occurred after injecting B16F10 (mouse melanoma cell line) into the paw pad of the mouse (FIG. 3). In addition, as a result of visualizing these RNA sequencing data in a volcano plot and presenting the data as log 2 fold-change, it was confirmed that the expression levels of IKZF1 and IKZF3 in the metastatic site were about 6-fold higher than those in the primary site, which means a statistically significant increase (FIG. 3).

Increased Expression Levels of IKZF1 and IKZF3 were Also Confirmed in Circulating Tumor Cells (CTCs).

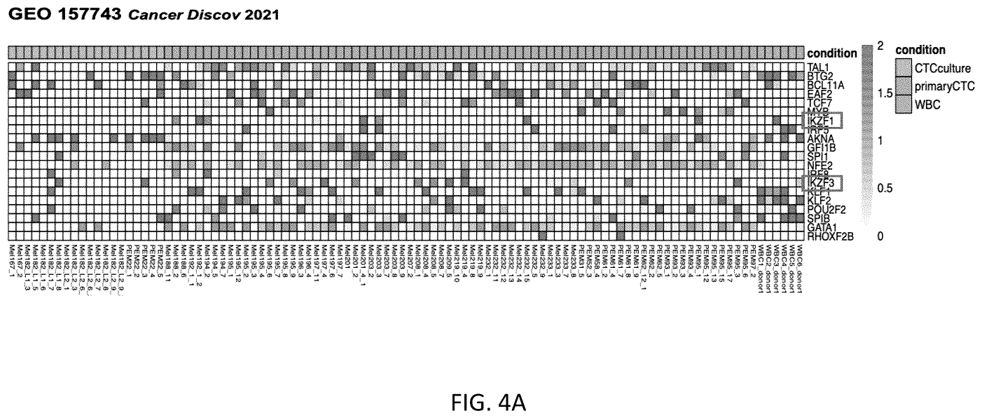

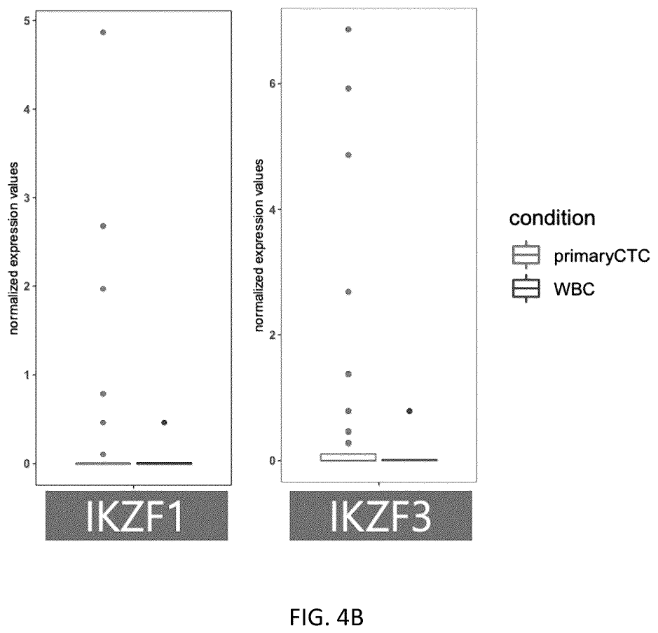

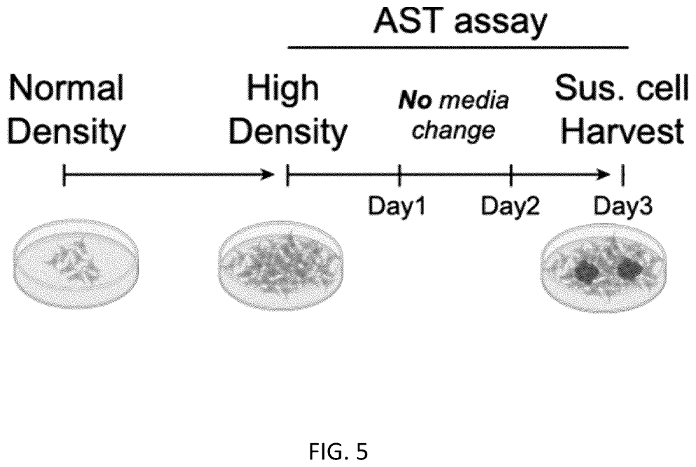

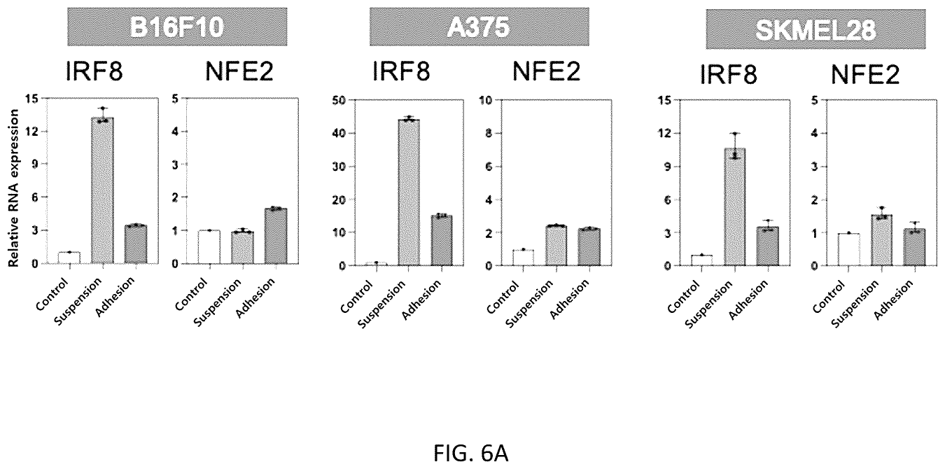

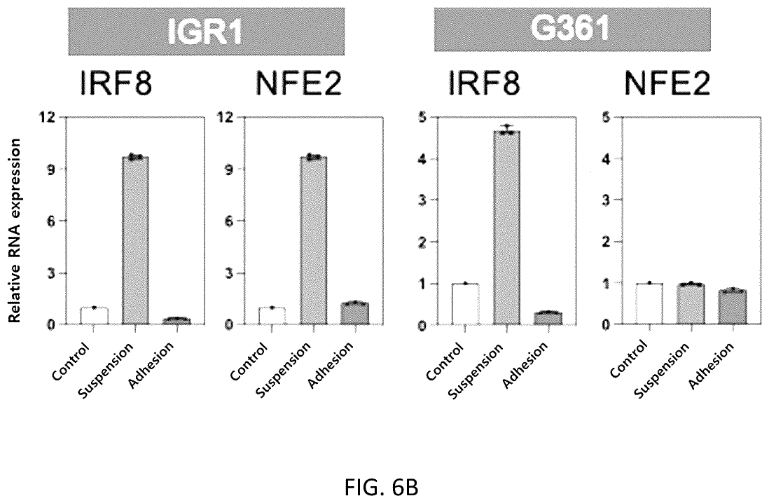

Most solid tumors originate from epithelial cells, and the well-known theory for the mechanism by which these solid cancer cells acquire metastatic ability is epithelial-to-mesenchymal transition (EMT) mechanism. Tumor cells that have undergone epithelial-to-mesenchymal transition invade and migrate to blood vessels, infiltrate the endothelial cells of the blood vessels, and enter the blood to form circulating tumor cells (CTCs). In order for tumor cells shed from the primary tumor and metastasize through the blood as described above, the transition of primary tumor cells, which are adherent cells, are required to transition to circulating tumor cells (CTCs), which are suspension cells. The present inventors examined whether the expression levels of IKZF1 and IKZF3 also increased in these circulating tumor cells (CTCs) (FIG. 4A).

As a result of isolating tumor cells from the blood of GEO157743 (Cancer Discov 11 (3): 678-695), a human melanoma patient tissue, and performing RNA sequencing of the tumor cells to determine the RNA expression levels of genes in the tumor cells (CTCs) from the patient's blood, it was confirmed that there was a group with higher relative expression levels of IKZF1 and IKZF3 than control white blood cells (WBCs) (FIG. 4B). This result shows that increased expression levels of IKZF1 and IKZF3 are highly correlated with metastasis of tumor cells.

### Increased Expression Levels of IRF8 and NFE2 are Also Confirmed Through RNA Sequencing Analysis.

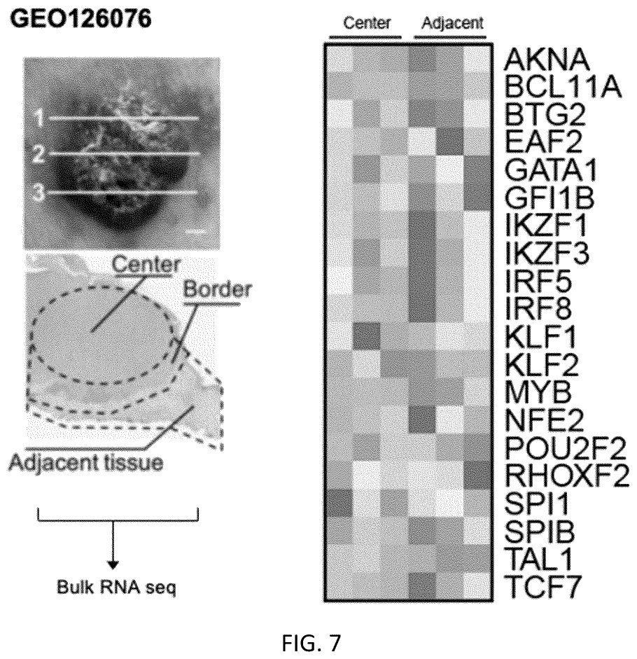

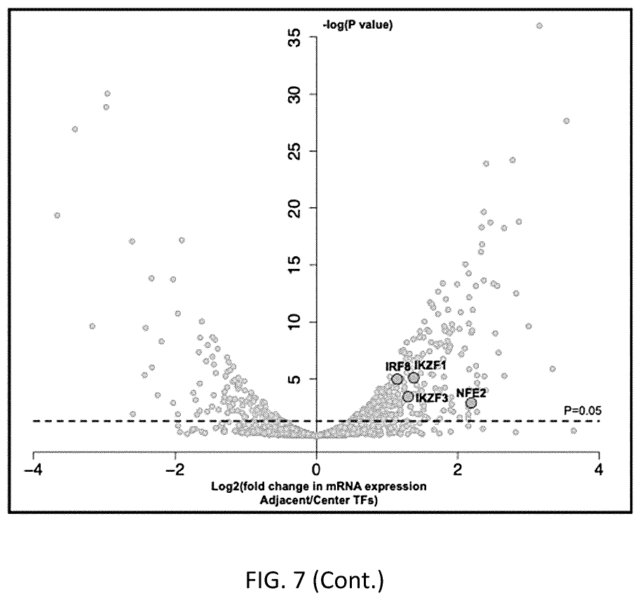

As a result of analysis performed using RNA sequencing data of GEO126076 (Cancer research 2019; 79 (10): 2736-2747), it was confirmed that the expression levels of IKZF1 and IKZF3, as well as NFE2 and IRF8, increased. These results were confirmed by dividing the melanoma patient's primary site into center, border, and adjacent tissues and then measuring the gene expression levels in each tissue through analysis of RNA sequencing data (FIG. 7). As a result of visualizing the RNA sequencing data in a volcano plot and presenting the data as log 2 fold-change, it was confirmed that the expression levels in the adjacent tissue were 1.5-fold higher in those in the center tissue, which means a statistically significant increase (FIG. 7).

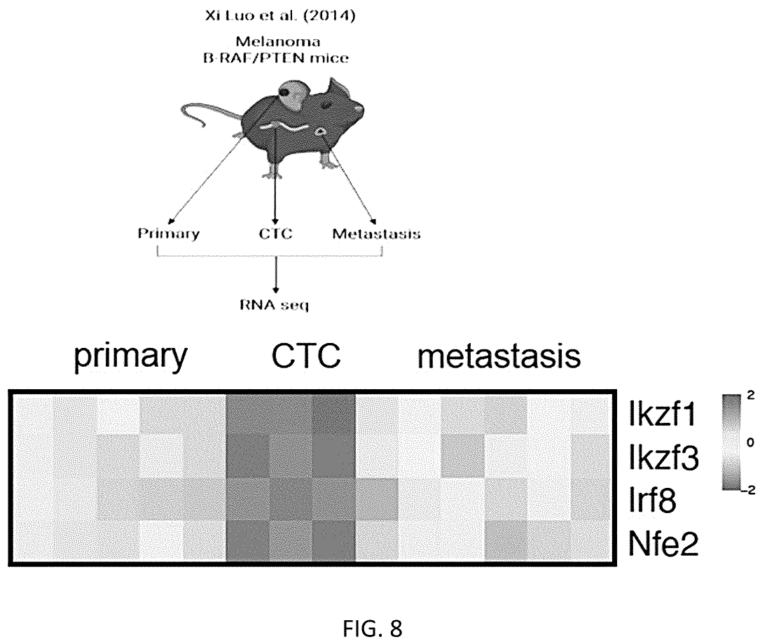

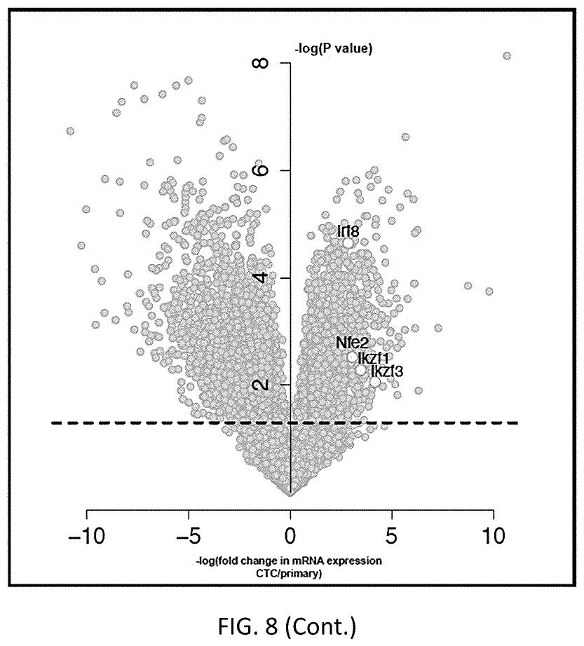

Analysis was performed using RNA sequencing data of GSE52031 (Cell report 2014 8; 7 (3): 645-53). For mice used, a model with a mutation in the BRAF/PTEN gene was used as a spontaneous melanoma mouse model. When metastasis occurred, data were obtained by RNA sequencing of each of the primary site, the blood circulating tumor cells (CTCs), and the metastatic site. From the data, it could be confirmed that the expression levels of IKZF1 and IKZF3, as well as NFE2 and IRF8, were higher in circulating tumor cells compared to the primary site, and the relative amount was about 6-fold higher when presented as log 2 fold-change, which is statistically significant (FIG. 8).

Increased Expression Levels of IRF8 and NFE2 were Also Confirmed in Circulating Tumor Cells (CTC).

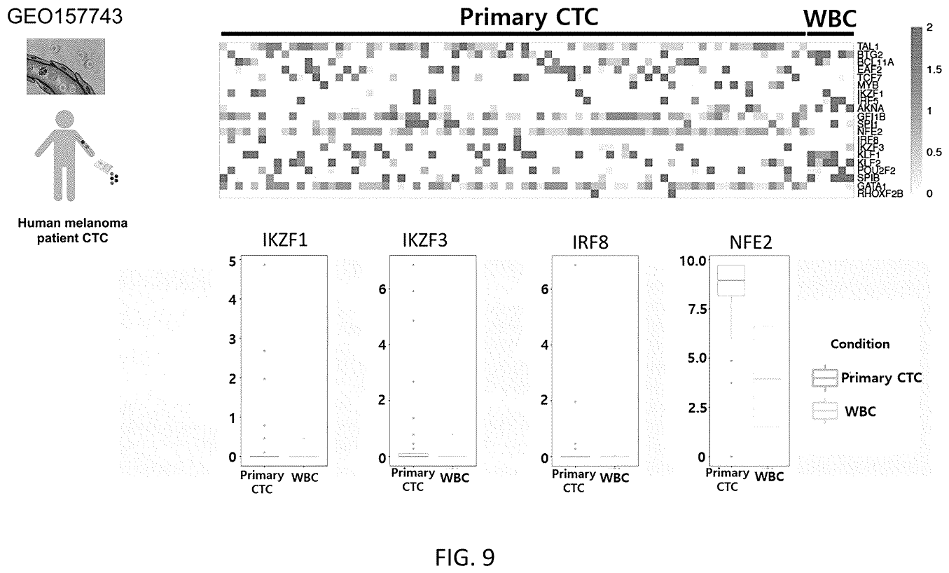

As a result of isolating tumor cells from the blood of GEO157743 (Cancer Discov 11 (3): 678-695), a human melanoma patient tissue, and performing RNA sequencing of the tumor cells to determine the RNA expression levels of genes in the tumor cells (CTCs) from the patient's blood, it was confirmed that there was a group with higher relative expression levels of IKZF1 and IKZF3, as well as NFE2 and IRF8, than control white blood cells (WBCs) (FIG. 9). This result shows that increased expression levels of IKZF1, IKZF3, NFE2 and IRF8 are highly correlated with metastasis of tumor cells.

It was Confirmed that the Expression Levels of IKZF1, IKZF3, NFE2, and IRF8 Increased in Circulating Tumor Cells (CTCs) Isolated from the Blood of a Mouse Melanoma Model.

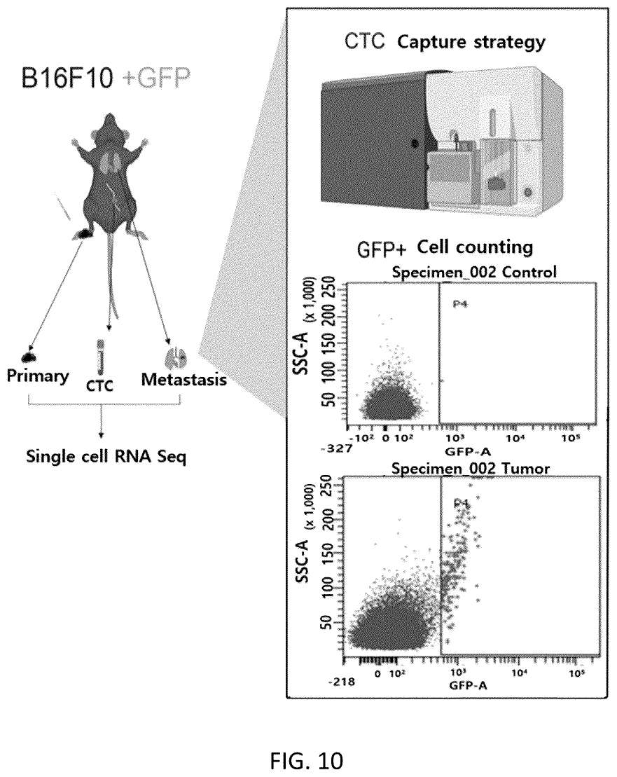

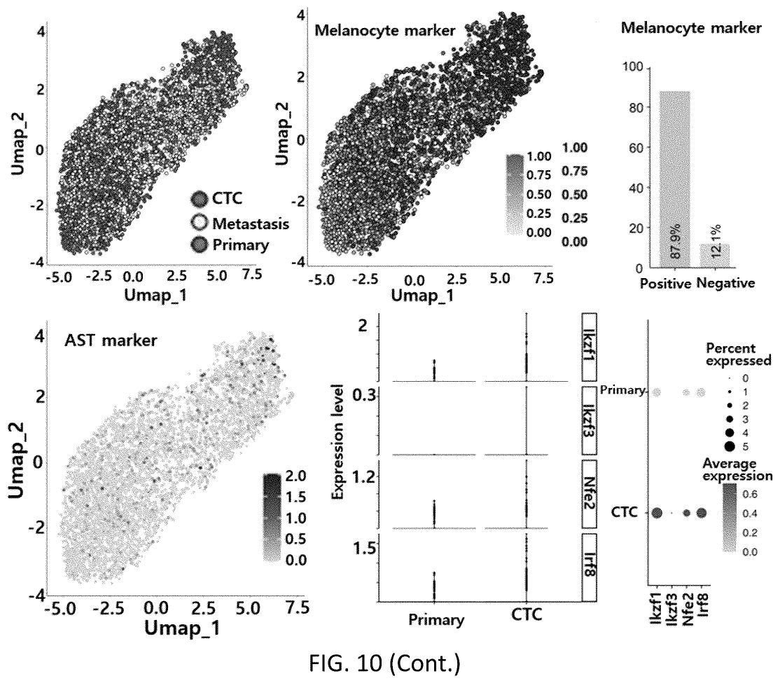

As a result of performing single-cell RNA sequencing after isolating only GFP+ tumor cells by FACS sorting from the primary site, the circulating tumor cells, and the metastatic site (lung), when lung metastasis occurred after injection of B16F10 (mouse melanoma cell line) into the paw pad of the mouse, it could be confirmed, through melanoma marker expression in single-cell RNA sequencing, that melanoma cells were well obtained, and it could be confirmed that the expression levels of IKZF1, IKZF3, NFE2, and IRF8 increased in circulating tumor cells compared to the primary site (FIG. 10).

It was Confirmed that the Efficiency of Transition to Suspension Cells Decreased in the IKZF1 Knock-Out B16F10 Cell Line.

The IKZF1 gene in the B16F10 cell line was knocked out (KO) using CRISPR technology. For the guide RNA used in CRISPR, as a result of observing the sequence and band size using PCR, it could be confirmed that the IKZF1 gene was properly knocked out by CRISPR technology (FIG. 11, left). As a result of performing AST assay on the IKZF1 KO cells versus control (CNT) cells to measure the efficiency of transition from adherent cells to suspension cells, it could be confirmed that the efficiency of transition from adherent cells to suspension cells (AST efficiency) decreased in the IKZF1 KO cells (FIG. 11, right).

It was Confirmed that Injection of an IKZF Inhibitor into a Melanoma Mouse Model Resulted in a Decrease in the Efficiency of Transition to Circulating Tumor Cells.

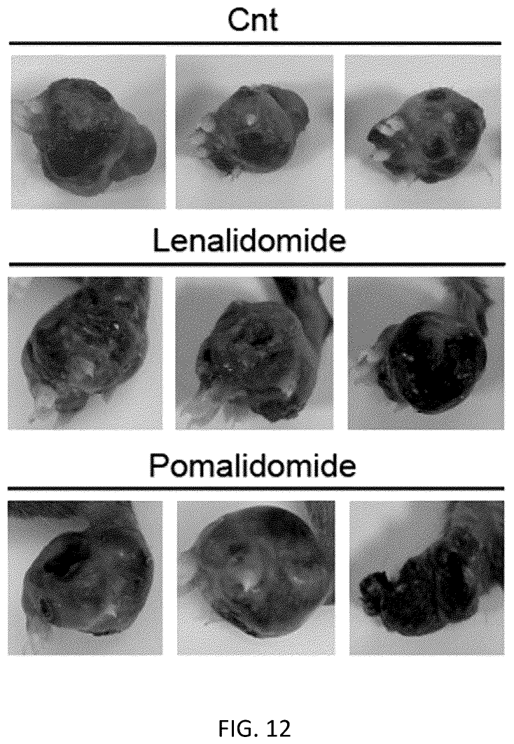

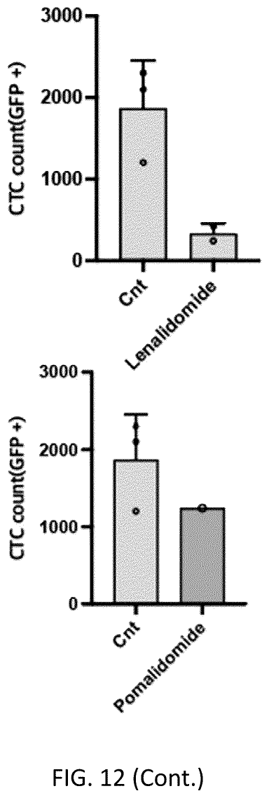

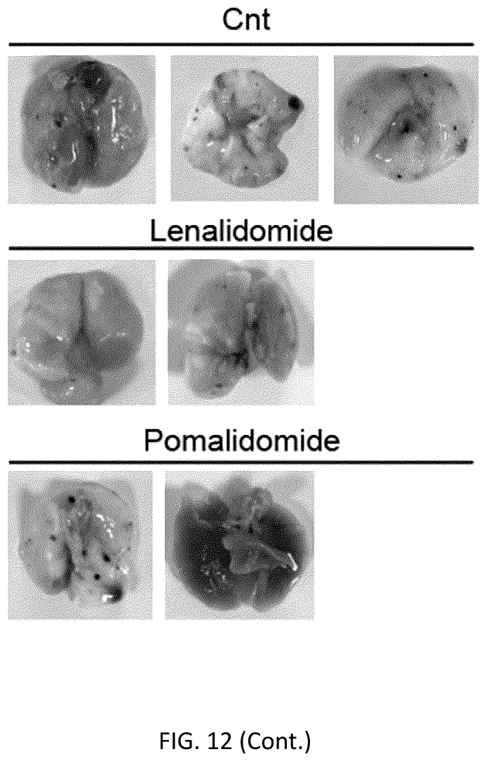

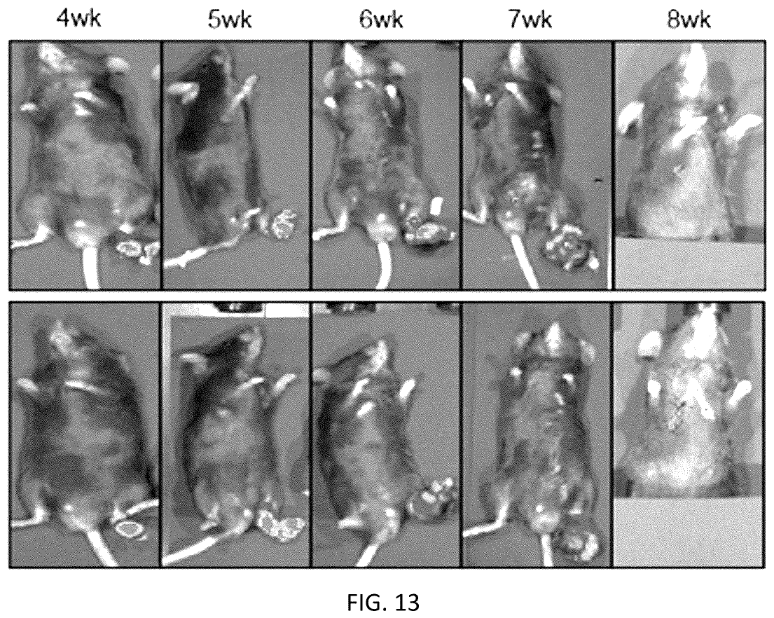

As a result of injecting lenalidomide or pomalidomide, a substance known to have a mechanism of inducing the degradation of IKZF1 and IKZF3, into a melanoma mouse model, and then counting circulating tumor cells, it could be confirmed that the number of circulating tumor cells decreased, suggesting that IKZF1 and IKZF3 are involved in the transition from adherent cells to circulating tumor cells (FIG. 12).

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1-11. (canceled)

12. A method for diagnosing metastatic cancer, the method comprising measuring an expression level of at least one protein selected from the group consisting of IKZF1 (Ikaros Transcription Factor 1), IKZF3 (Ikaros Transcription Factor 3), NFE2 (Nuclear Factor, Erythroid 2), and IRF8 (Interferon Regulatory Factor 8), or a gene encoding the protein.

13. The method according to claim 12, wherein measuring the expression level of the gene encoding the protein is carried out using a primer or probe that binds specifically to a nucleic acid molecule of the gene.

14. The method according to claim 12, wherein measuring the expression level of the at least one protein is carried out using an antibody or an antigen binding-fragment thereof that binds specifically to the protein or an aptamer that binds specifically to the protein.

15. The method according to claim 12, wherein the cancer is skin cancer.

16. The method according to claim 15, wherein the skin cancer is melanoma.

17. A method for providing information necessary for diagnosing metastatic cancer, the method comprising a step of measuring an expression level of at least one protein selected from the group consisting of IKZF1, IKZF3, NFE2, and IRF8, or a gene encoding the protein, in a biological sample isolated from a subject.

18. A method for preventing or treating metastatic cancer, the method comprising administering, as an active ingredient, a composition comprising an inhibitor of at least one selected from the group consisting of IKZF1, IKZF3, NFE2, and IRF8 to a subject in need thereof.

19. The method according to claim 18, wherein the cancer is skin cancer.

20. The method according to claim 19, wherein the skin cancer is melanoma.

21. A method for screening a composition for preventing or treating metastatic cancer, the method comprising steps of:
(a) bringing a test substance into contact with a biological sample containing at least one protein selected from the group consisting of IKZF1, IKZF3, NFE2 and IRF8, a gene encoding the protein, or cells expressing the protein or gene; and
(b) measuring an expression level of the at least one protein or the gene encoding the protein in the biological sample,
wherein, when the expression level of the at least one protein or the gene encoding the protein decreases, the test substance is determined to be a composition for preventing or treating metastatic cancer.

22. The method according to claim 21, wherein the biological sample contains tumor tissue or tumor cells.
